(19) United States
(12) Patent Application Publication
(10) Pub. No.: US 2025/0344727 A1
(43) Pub. Date: Nov. 13, 2025

(54) AN EDIBLE PRODUCT, FROM CULTIVATED CELLS, WITH AN ENHANCED MEAT-LIKE FLAVOR AND PRODUCTION METHOD THEREOF

(71) Applicant: SUPRÊME, Evry-Courcouronnes Cedex (FR)

(72) Inventors: Nadine BONGAERTS, Evry-Courcouronnes Cedex (FR); Victor SAYOUS, Evry-Courcouronnes Cedex (FR); Marion GAFF, Evry-Courcouronnes Cedex (FR); Gemma LYONS, Evry-Courcouronnes Cedex (FR)

(21) Appl. No.: 18/854,309

(22) PCT Filed: Apr. 7, 2023
(86) PCT No.: PCT/EP2023/059343

§ 371 Date: Oct. 4, 2024

(30) Foreign Application Priority Data
Apr. 7, 2022 (EP) 22305486.7

(51) Int. Cl. A23L 13/40

(52) U.S. Cl. CPC A23L 13/48; C12Y 101/03004; C12Y 115/01001

(57) ABSTRACT

The invention relates to an edible product, produced from cultivated cells, having meat-like flavor and a Method (100) of producing, from cultivated cells, an edible product having meat-like flavor, the method (100) comprising the steps of: —Providing (110) a food matrix, said food matrix comprising cultivated cells and/or cultivated cells extracts, said cultivated cells are cells from an organism of the Animalia kingdom excluding human, cells from an organism of the Bacteria kingdom and/or cells from an organism of the Fungi kingdom; —Treating (140) the food matrix with a reactive oxygen species (ROS) producing enzyme, to obtain a treated food matrix; and Processing (160) the treated food matrix to produce an edible product having a meat-like flavor, said processing including an inactivation and/or a removal of the ROS producing enzyme.

### FIELD OF THE INVENTION

The present invention relates to the field of alternatives to meat products and to meat product derivatives. In particular, the invention relates to the field of meat substitute. This invention provides a new edible product with an enhanced meat-like flavor and a production method thereof. This edible product with an enhanced meat-like flavor can be considered as a substitute for meat. For example, this edible product can be a foie-gras substitute, not obtained from force-fed animals, with a strong and pleasant foie gras flavor.

### DESCRIPTION OF RELATED ART

Traditional meat production is a resource-intensive process that generates a significant environmental footprint. Domesticated animals are raised in agricultural settings requiring substantial quantities of fresh water, feed, land, and other resources. (Mark J Post: “Cultured meat from stem cells: Challenges and prospects” Meat science, Elsevier Science, GB, vol 92, no 3, 3 Apr. 2012, pages 297-301). Food production is considered as responsible for approximately 26% of global GHG emissions and livestock & fisheries account for 31% of those food emissions.

In another aspect, animal welfare is becoming a growing concern. For example, the European Union has put in place various laws concerning animal welfare since 1986. Various directives provide rules for the protection of laying hens (in 1986 and 1988), calves and pigs (in 1991), and in 1998 the directive 98/58/EC of the Council on the protection of animals kept for farming purposes has established general rules concerning the protection of animals, whatever the species.

Several meat substitutes have been developed, such as substitutes from insects, plant-based components and/or cultured meat based on cell technology. Although technologies to mimic the mouthfeel and appearance of these products are continuously improving, it appears difficult to mimic complex flavor profiles of meat with proteins, sugars and fats from insects, plants, or cell technology origin.

In particular, cell cultivation technologies are rapidly developing to respond to the new demand of consumers of alternatives to meat production that ensures animal welfare. While the replacement of certain products could be essential in relation to environmental or ethical issues (with regard to animal welfare), alternative production technologies face difficulties in the reproduction of certain complex flavors. The creation of meat flavor is achieved through providing heat (cooking) to an intricate balance of lipid and water-soluble precursor metabolites. Such metabolites are products of a myriad of metabolic processes occurring both in the lifetime of the animal and in the conversion of flesh to meat after slaughter. To further confound the complexity of flavor, molecules that elicit taste and flavor have varying thresholds of detection and only when present together in the right combination and at the correct concentration can provide the target profile of interest. This is especially the case for meat products that result from specific feeding regimens that evoke the correct conditions for a distinct set of molecules to form, such as the force-feeding of palmiped to make “foie gras”, the long feeding periods that are necessary to produce fine meat such as Wagyu beef meat or meat products that undergo post-mortem aging treatments.

Foie gras is a specialty food product made of the liver of a duck or a goose. Foie gras is defined as the liver of a duck or a goose fattened through force-feeding, known as gavage. Ducks are force-fed twice a day for 12.5 days and geese three times a day for around 17 days. Ducks are typically slaughtered at 100 days and geese at 112 days. Unlike natural fattening, which is authorized everywhere in the world, force-feeding is prohibited in several countries, either under general animal protection laws or because of specific prohibitions. In particular, it is prohibited in Argentina, California (United States), Israel, Norway, Switzerland, Turkey, as well as in most countries of the European Union, where it is now only practiced in five countries (France, Hungary, Bulgaria, Spain and Belgium).

Food flavor perception results from a combination of hundreds of compounds that are detected in the nose, throat and mouth while eating. It has been shown that slight modifications of compounds can alter the eating experience, for example the lipids profiles induced by feeding protocol (force-feeding compared to spontaneous fattening) can have a significant impact on flavor and appearance (Fernandez X. et al, Comparison of the composition and sensory characteristics of goose fatty liver obtained by overfeeding and spontaneous feeding, Poultry science, 2019, p 6149-6160).

It has been well-proven that while aqueous-derived (water phase) molecules provide general meat flavor, lipid-derived molecules provide species-specific flavor that creates identifiable aroma and taste (Massimino W. et al, Ontogeny of hepatic metabolism in mule ducks highlights different gene expression profiles between carbohydrate and lipid metabolic pathways, BMC Genomics, 2020 and Wei R. X. Overfeeding influence on antioxidant capacity of serum, liver, gut, and breast muscle in Gang Goose and Tianfu Meat Goose, Poultry Science, 2020, 455-464), in which lipid oxidation is both generating flavor as well as a major cause of reducing the quality of food. Controlling lipid oxidation is considered as a challenge in the food industry particularly with heated fatty products.

During the past 20 years, a significant amount of research has been dedicated to understanding the biochemical basis of postmortem aging and flavor, largely in the context of meat from ruminant, fish and poultry (Virginia C. Resconi et al. The Development of Aromas in Ruminant Meat. Molecules. 2013 June; 18 (6): 6748-6781; Spanier A. M. et al. The effect of post-mortem aging on meat flavor quality in Brangus beef. Correlation of treatments, sensory, instrumental and chemical descriptors. Food Chemistry Volume 59, Issue 4, August 1997, Pages 531-538; Muhammad Sajid Arshad et al. Ruminant meat flavor influenced by different factors with special reference to fatty acids. Lipids Health Dis. 2018; 17:223). For example, it has been shown that different aging methods have an effect on the changes in the concentrations of aroma volatiles of beef. Most changes in the concentrations of aroma volatiles of dry-aged beef were associated with propanal, 2-methylbutanal, 2-methylpropanal, 1-butanamine, trimethylamine, 2-methyl-2-propanethiol, and ethyl propanoate, which were mainly produced by lipid oxidation and/or microbial activity (e.g., proteolysis and lipolysis) during the dry aging period (Dongheon Lee et al. Effect of Different Aging Methods on the Formation of Aroma Volatiles in Beef Strip Loins; Foods. 2021 January; 10 (1): 146). Another recent study shows that more glutamate-containing dipeptides and nucleotides were found in dry-aged beef and that dry-aging lowered metabolites related to undesirable flavor such as terpenoids (Derico Setyabrat et al. Elucidating mechanisms involved in flavor generation of dry-aged beef loins using metabolomics approach. Food Research International; Volume 139, January 2021, 109969).

Mimicking all complex flavors naturally produced in vivo by ex vivo systems is extremely difficult. The product, as a meat substitute, should mimic the aesthetic and organoleptic qualities of meat such as size, appearance, flavor and texture, to increase people's willingness to consume meat replacement. (Macdiarmid et al., 2016, Eating like there's no tomorrow: Public awareness of the environmental impact of food and reluctance to eat less meat as part of a sustainable diet. Appetite. 2016 Jan. 1; 96:487-493). Hence, there is a real need to improve the overall acceptance, appetizing of meat substitutes without relying on the exclusive use of chemical additives like aroma. For instance, US patent application 2020/0392461 relates to food products derived from avian cells produced in vitro and methods of cultivation of avian cells in low serum or the absence of serum.

In particular, there are important needs for processes capable of producing a meat substitute, such as a foie-gras substitute, which has comparable or enhanced organoleptic and nutritional qualities. Such a meat substitute shall mimic the complexity of meat flavor, such as foie gras flavor, while demonstrating cooking performances similar to the one expected for a high quality product such as conventional foie gras.

### SUMMARY OF THE INVENTION

The following sets forth a simplified summary of selected aspects, embodiments and examples of the present invention for the purpose of providing a basic understanding of the invention. However, this summary does not constitute an extensive overview of all the aspects, embodiments and examples of the invention. Its sole purpose is to present selected aspects, embodiments and examples of the invention in a concise form as an introduction to the more detailed description of the aspects, embodiments and examples of the invention that follow the summary.

The invention aims to overcome the disadvantages of the prior art. In particular, the invention proposes a method of producing, from cultivated cells, an edible product having a meat-like flavor, the method comprising the steps of:
Providing a food matrix, said food matrix comprising cultivated cells and/or cultivated cells extracts, said cultivated cells are cells from an organism of the Animalia kingdom excluding human, cells from an organism of the Bacteria kingdom and/or cells from an organism of the Fungi kingdom;
Treating the food matrix with a reactive oxygen species (ROS) producing enzyme, to obtain a treated food matrix; and
Processing the treated food matrix to produce an edible product having a meat-like flavor, said processing including an inactivation and/or a removal of the ROS producing enzyme.

Such a method for the preparation of an edible product having meat-like flavor allows to avoid the slaughtering of animals. As it will be described in detail, such a method is simple and sustainable compared to standard methods. In addition, such a method can be used for the production of an edible product having meat-like flavor from cultivated cells without relying exclusively on synthetical aroma or artificial flavor. The edible product being thus an alternative to conventional meat derived product. The development of flavor over time in traditional meat occurs due to the biochemical reactions induced in feeding, slaughter, processing and maturation. By applying the present invention, the complexity of these reactions can be achieved in a matter of minutes.

As mentioned, lipid oxidation is a major cause of reducing the quality of food and its control is considered as a challenge in the food industry. As it will be described, compared to the use of ROS, the use of a ROS producing enzyme allows the gradual generation of a low concentration of ROS in the food matrix and this allows the generation of complex flavors without a deterioration of the product.

Moreover, as it will be described hereafter, when the food matrix comprises cultivated duck cells, it allows the production of an edible product with foie gras flavor without the slaughtering and/or force-feeding of ducks or geese. Hence, in some aspect, the invention allows the production of foie gras substitute from raw materials produced in accordance with animal welfare, in particular without force-feeding of animals; said foie gras displays high quality in olfactory, gustatory and tactile characteristics (flavor), while tasting, similar to those of force-feeding-based foie gras. Also, such an edible product can mimic a large variety of products such as highly marbled meat or fatty fish flesh such as salmon flesh.

According to other optional features of the method according to the invention, it can optionally include one or more of the following characteristics alone or in combination:
said processing step comprises an inactivation, the inactivation being a heat treatment, a modification of pH and/or a modification of the ionic strength of the treated food matrix. Preferably, the inactivation is a heat treatment of the treated food matrix at a temperature over 40° C. for more than 30 sec.
the processing step comprises a removal of the ROS producing enzyme. This is particularly adapted when the method comprises the use of an immobilized ROS producing enzyme that can be incubated with the food matrix to be treated. This allows to reuse the enzyme and finely control the reaction. This is also particularly advantageous when carrying out the method in a continuous process. Hence, the ROS producing enzyme can be an enzyme already used for the treatment on another food matrix.
the ROS producing enzyme is a synthetic ROS producing enzyme. Preferably, the synthetic ROS producing enzyme produces at least one of the following reactive oxygen species: peroxides such as hydrogen peroxide, superoxide, hydroxyl radicals, singlet oxygen, nitric oxide, peroxinitrite, free radicals, hypochlorite or hypochlorous acid, peroxyl radicals such as alkylperoxyl and hydroperoxyl, alkoxyl radicals, or combination thereof. More preferably, the ROS producing enzyme, such as the synthetic ROS producing synthetic enzyme, produces hydrogen peroxide. For example, the ROS producing enzyme is an enzyme selected from: superoxide dismutase, glucose oxidase, cholesterol oxidase, lipases or combination thereof. These enzymes are particularly relevant to induce a meat-like flavor in the edible product.
the step of treating the food matrix comprises adding the ROS producing enzyme at a concentration over 0.01 IU/g of the food matrix in wet weight, preferably at a concentration over 0.1 IU/g of the food matrix in wet weight. The ROS producing enzyme can be added, at such concentration to the food matrix, in free or grafted form, for example grafted to walls or to electromagnetic substrate (e.g. nanoparticles, particles, ball, wall . . . ).
the reactive oxygen species producing enzyme is in contact with the food matrix during at most ten hours before being inactivated or removed during the processing step. The development of flavor over time in traditional meat occurs due to the biochemical reactions induced in feeding, slaughter, processing and maturation. By applying our method, we achieve the complexity of these reactions in a matter of minutes.
when treating the food matrix, said food matrix comprises at least 5% in weight of cultivated cells and/or cultivated cells extracts, from an organism of the Animalia kingdom excluding human, with respect to the wet weight of food matrix, preferably said cultivated cells are selected from: stem cells, cells derived from differentiation of non-human embryonic stem cells; cells derived from differentiation of non-human induced pluripotent stem cells; cells derived from transdifferentiated non-human isolated cells; immortalized mature non-human cells; differentiated cells derived from differentiation of non-human progenitor cells, fibro-adipogenic progenitors, muscle cells, hepatocytes, fibroblasts, adipocytes, chondrocytes, keratinocytes, and combination thereof. Without being limited by the theory, the presence of animal cultivated cells and/or cultivated cells extracts improves the ability of the food matrix to respond to the identified needs.
the food matrix comprises non-human cultivated hepatocytes, preferably said non-human cultivated hepatocytes are selected from: hepatocytes derived from differentiation of non-human embryonic stem cells; hepatocytes derived from differentiation of non-human induced pluripotent stem cells; hepatocytes derived from transdifferentiated non-human isolated cells; immortalized mature non-human hepatocytes; differentiated hepatocytes derived from differentiation of non-human progenitor cells.
the food matrix comprises at least 5% in weight of cultivated Mammalia cells excluding human, cultivated Aves cells, cultivated Actinopterygii cells, cultivated Malacostraca cells, combination thereof, and/or cultivated cells extracts thereof.
the food matrix further comprises at least 2% in weight of plant-based material, for example plant cells. The food matrix can further comprise at least 2% in weight of fermentation product, for example fermentation-based fat or fermentation-based protein.
prior to, or during the step of treating, the food matrix is homogenized with fat, preferably said fat being a non-human animal fat, a plant fat, a fermented fat or a mixture thereof. Preferably, it is during the step of treating that the food matrix is homogenized with fat. Without being limited by the theory, the presence of fat, in an emulsion, improves the ability of the food matrix to respond to the identified needs. Advantageously, the homogenization is carried out according to parameters allowing the formation of an emulsion, preferably of a microemulsion.
when treating the food matrix, said food matrix comprises at least 5% of lipids in weight with respect to the food matrix wet weight. The lipids can be found in the food matrix in different forms. They are preferably found in the form of triglycerides. In particular, when treating the food matrix, said food matrix comprises at least 1% in weight of triglycerides compared to the food matrix wet weight, preferably 2% of triglycerides in weight, more preferably 5% of triglycerides in weight, even more preferably at least 10% of triglycerides in weight, with respect to the food matrix wet weight.
when treating the food matrix, said food matrix comprises at least 1% in weight of unsaturated fatty acids, with respect to the food matrix wet weight. Without being limited by the theory, the presence of unsaturated fatty acids improves the ability of the food matrix to respond to the identified needs.
it comprises, after the step of treating the food matrix, a step of heat treatment until a core temperature of the food matrix of at least 50° C. is reached for at least 5 min. The step of heat treatment is preferably after the step of treating the food matrix, said step of heat treatment being before and/or after the processing step. The heat treatment can be conducted even if a removal of the ROS producing enzyme has been done.

According to another aspect, the invention can also relate to an edible product having a meat-like flavor, produced from cultivated cells, and obtainable by a process according to the invention, said edible product comprising a treated food matrix, said treated food matrix having been treated with a reactive oxygen species (ROS) producing enzyme, said treated food matrix comprising cultivated cells and/or cultivated cells extracts, said cultivated cells are cells from an organism of the Animalia kingdom excluding human, cells from an organism of the Bacteria kingdom and/or cells from an organism of the Fungi kingdom; and a reactive oxygen species (ROS) producing enzyme.

According to other optional features of the method according to the invention, it can optionally include one or more of the following characteristics alone or in combination:
the edible product comprises at least 1% in weight of the cultivated cells and/or extracts thereof, with respect to the total edible product wet weight and it further comprises the ROS producing enzyme at a concentration of at least 0.0001 ng/g, preferably of at least 0.001 ng/g. Alternatively, it comprises the ROS producing enzyme at a concentration of at least 0.005 IU/g of the edible product in wet weight.
the edible product comprises the treated food matrix as an emulsion, preferably a microemulsion. Advantageously, the edible product comprises a food grade oil-in-water (O/W) or water-in-oil (W/O) emulsion or microemulsion.
the edible product comprises at least 0.5% in weight of unsaturated fatty acids, with respect to the total edible product wet weight. Preferably, the edible product comprises at least 0.5% in weight of unsaturated fatty acids in the form of triglycerides.

### BRIEF DESCRIPTION OF THE DRAWINGS

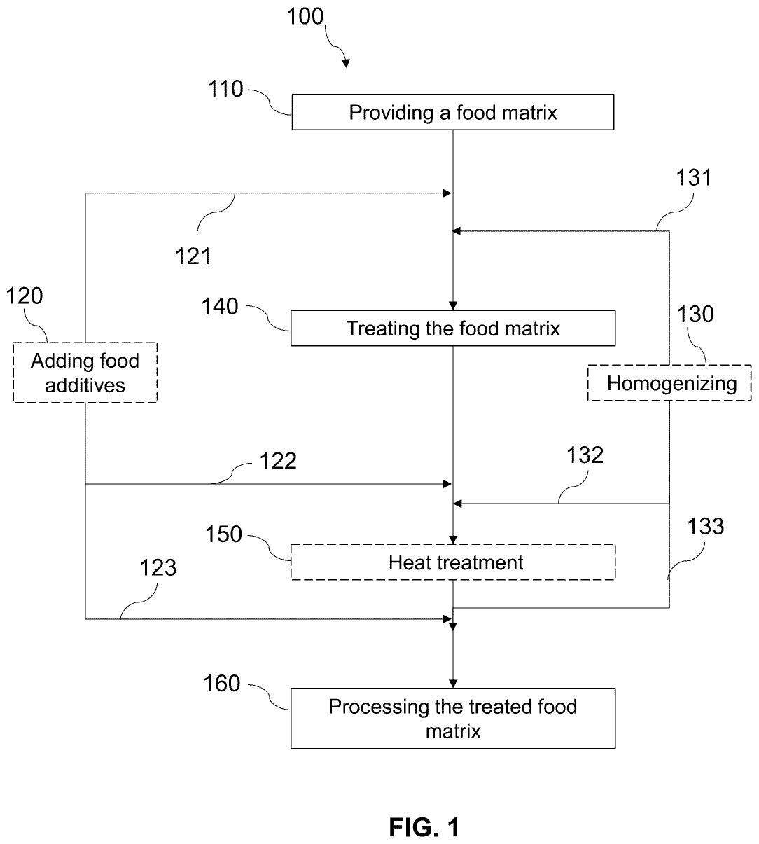

The foregoing and other objects, features and advantages of the present invention will become more apparent from the following detailed description when taken in conjunction with the accompanying drawings in which FIG. 1 is a schematic view of a method of producing an edible product with a meat-like flavor according to an embodiment of the invention.

Several aspects of the present invention are disclosed with reference to flow diagrams and/or block diagrams of methods and devices.

On the FIGURE, the flow diagrams and/or block diagrams show the architecture, the functionality and possible implementation of devices or systems or methods, according to several embodiments of the invention.

In some implementations, the functions associated with the box may appear in a different order than indicated in the drawings. For example, two boxes successively shown, may be performed substantially simultaneously, or boxes may sometimes be performed in the reverse order, depending on the functionality involved.

### DETAILED DESCRIPTION

A description of example embodiments of the invention follows.

The expression “edible product” as used herein can relate to a product suitable for animal consumption and preferably a product intended for human consumption. An edible product according to the invention can be a ready to eat (i.e. finalized) food product or an intermediate in the production chain of a finalized food product. An edible product according to the invention can be produced in the form of a snack which may be pressed, fried and/or toasted; a sauce; a spread; a pasta; a paste; transformed meat-analogues or food specialty food such as sausage or cured sausage, paté or foie gras; a meat dough; a soup; a smoothie; a seafood; untransformed meat-analogues such as “flesh like” products.

In the following description, the term “meat” can refer to any edible part of an animal such as animal tissues taken from a dead animal. Hence, a meat can refer to offals such as liver tissues, fat tissues, muscles tissues, retrieved from a dead animal. The dead animal can refer to all species of the Animalia kingdom excluding human and preferably to all edible species such as a non-human vertebrate, for example, livestock, fish, birds; insects; a crustacean, for example a shrimp, prawn, crab, crayfish, and/or a lobster; a mollusk, for example an octopus, squid, cuttlefish, scallops, snail. Hence, for example, the invention allows the production of an edible product such as a product mimicking foie gras, marbled beef, or salmon flesh.

As used herein, a “flavor” is generally the quality of the product that affects the sense of taste and/or the aroma. Hence, a “meat-like flavor” can refer to a flavor which is close, or which approximates, the flavor of the related conventional meat product. Hence, an edible product with a meat-like flavor can be considered as an alternative to a meat product.

As used herein, the expression “reactive oxygen species” (ROS) can be considered as referring to derivatives of molecular oxygen that occur as a normal attribute of aerobic life. Examples of ROS include peroxides, superoxide, hydroxyl radical, singlet oxygen, and alpha-oxygen.

As used herein, the expression “in weight” is generally referring to the weight of something compared to the weight of the food matrix or the edible product, either the wet weight or the dried weight can be considered. Preferably, percentages are disclosed in reference to the wet weight.

As used herein, the expression “core temperature” can be considered as referring to the temperature in the center of the food. It can for example be measured with a penetration thermometer.

As used herein, “a food matrix” can relate to a matrix, wet or dried, suitable for human consumption. A food matrix is constituted mainly of lipids, proteins and/or carbohydrates. When a composition is referring to the weight of the food matrix, either the wet weight or the dried weight can be considered. Preferably, percentages are disclosed in reference to the wet weight. The relative moisture content of a wet food matrix can be of 10% or over. For example, the relative humidity of a wet food matrix can range from 20 to 95%. Preferably, the moisture content of a wet food matrix can range from 30 to 80%. A food matrix can comprise plant material obtained from edible plants, including the flowers, fruits, stems, leaves, roots, and seeds. For example, a food matrix can comprise at least 20% in weight of plant material, preferably at least 30% in weight of plant material, more preferably 40% in weight of plant material, even more preferably 50% in weight of plant material.

As used herein, the expressions “cultivated cells” or “cultured cells” are used interchangeably. They can refer in particular to cells with a growth controlled by mankind, for example in an industrial process, e.g. using a culture medium, as opposed to cells from meat that have been multiplied in a living organism or cells grown in a natural environment (e.g. forest grown mushrooms). Cultivated cells can refer to cells belonging to Animalia kingdom but also to Bacteria and Fungi kingdoms. Hence, a food matrix can refer to a matrix comprising cultivated cells. Cultivated cells can be cultured from cells of any origin such as cells from a biopsy sample, derived from stem cells or correspond to stem cells themselves.

As used herein, the expression “extracts of cultivated cells” can refer to any fraction of disrupted cells or to any biological material purified or partially purified recovered from disrupted cells. Disrupted cells can be cells having partially or completely destroyed cell walls. In a food matrix, extracts of cultivated cells can comprise both disrupted cells and/or biological material recovered from disrupted cells.

As used herein, the terms “improved” or “optimized” refer to qualities at least equal to those of a conventional meat product, usually obtained by animal farming, animal slaughtering or animal force-feeding.

The term “about” as used herein can allow for a degree of variability in a value or range, for example, within 10%, within 5%, or within 1% of a stated value or of a stated limit of a range.

The term “substantially” as used herein refers to a majority of, or mostly, as in at least about 50%, 60%, 70%, 80%, 90%, 95%, 96%, 97%, 98%, 99%, 99.5%, 99.9%, 99.99%, or at least about 99.999% or more.

As mentioned, the production of tasty meat-like product without animal slaughtering or foie gras without force-feeding is a challenge for both animal welfare and the environment. In addition to animal welfare or environmental protection, it appears necessary to produce a product which corresponds to the expectations of consumers by exhibiting improved organoleptic and nutritional properties; close to those of a conventional product without a degraded lipid profile or extensive oxidation.

A new method has been developed for the production of an edible product having a meat-like flavor without making use of any tissues from a slaughtered animal and for example an edible product with foie gras flavor, that does not comprise hepatocytes obtained from a force-fed animal.

Indeed, a specific treatment of non-human cells has been developed to produce an edible product having meat-like and complex flavor and in particular an edible product with foie gras flavor, for which flavors, and “foie-gras” flavor are greatly improved.

Such method is of particular interest when used on cultivated cells. Indeed, a continuous and homogeneous source of cells can be prepared to produce an edible product, as an edible food product, with enhanced meat-like flavor with no more need of animal slaughtering and/or force feeding.

In particular and as it will be detailed in example, it has been developed a method based on a specific oxidation to create complex flavors while other methods based on oxidation would lead to deterioration of the product and its organoleptic properties.

Hence, according to a first aspect, the invention relates to a method 100 of producing an edible product having meat-like flavor.

Advantageously, the edible product according to the invention can be considered as an alternative or substitute to conventional meat product. An edible product can for example be a finished food product that can be consumed directly, or eventually after a cooking step and/or processing step (crushing, squishing, cutting, grinding, mixing, shredding, squeezing, dosing, molding, pressing, 3D printing, extruding baking or cooking steps such as smoking, roasting, frying, surface treatment, coating). In particular, the edible product can be an alternative product to the meat which aims to imitate a known meat product, transformed or not (e.g. steak, sausage, pâté . . . ).

However, an edible product can also correspond to an ingredient for use in the preparation of a ready to eat food product. For example, an edible product according to the invention can be in the form of a liquid (e.g. suspension, solution or emulsion), granulate or powder which can be used in the preparation of a ready to eat food product such as an alternative to a meat product.

As explained, an edible product according to the invention has a meat-like flavor, preferably it has a flavor of meat more pronounced than the sum of the meat flavors of the products composing it. In particular, as it will be illustrated in example, an edible product having meat-like flavor can exhibit an enhanced meat-like flavor compared to an edible product produced from non-treated cultivated cells. Moreover, a product according to the invention having a meat flavor can display complex meat flavor compared to non-treated product, mimicking meat flavor obtained with cells grown in vivo.

Preferably, the edible product having a meat-like flavor is obtained from cultivated cells that are intact in the edible product or that have been disrupted. Hence, as it will be detailed hereafter, the edible product having a meat-like flavor according to the invention can comprise intact cultivated cells, disrupted cultivated cells, extracts of cultivated cells or a combination thereof.

In particular, as illustrated in FIG. 1, a method of producing an edible product having a meat-like flavor according to the invention will comprise the following steps: providing 110 a food matrix, treating 140 the food matrix with a reactive oxygen species (ROS) producing enzyme, and processing 160 the treated food matrix to produce an edible product having meat-like flavor, which preferably include inactivating or removing the ROS producing enzyme.

A method according to the invention can also comprise the following steps: a step of adding a food additive 120, a step of homogenization 130, and a step of heat treatment 150.

As shown in FIG. 1, a method 100 of producing an edible product having meat-like flavor according to the invention comprises a step of providing 110 a food matrix.

As mentioned, and illustrated in example, the inventors developed a solution capable of generating a meat-like flavor in a food matrix comprising cells when treated according to a method of the invention. Said cells belong to, or originate from, the Animalia kingdom excluding human, the Bacteria kingdom and/or the Fungi kingdom.

Hence, preferably, the food matrix comprises cells and/or cells extracts, said cells being cells from an organism of the Animalia kingdom excluding human, cells from an organism of the Bacteria kingdom and/or are cells from an organism of the Fungi kingdom. More preferably, the food matrix comprises cells and/or cell extracts, said cells being cultivated cells from an organism of the Animalia kingdom excluding human, cultivated cells from an organism of the Bacteria kingdom and/or cultivated cells from an organism of the Fungi kingdom. As mentioned, cells can be intact cells and/or disrupted cells.

Even more preferably, the food matrix comprises cultivated cells from an organism of the Animalia kingdom excluding human or cells extracts thereof.

The food matrix can comprise at least 1% in weight, with respect to a total weight of the food matrix, of cultivated cells from an organism of the Animalia kingdom excluding human, cultivated cells from an organism of the Bacteria kingdom and/or cultivated cells from an organism of the Fungi kingdom, and/or cells extracts thereof. Preferably, the food matrix comprises at least 2% in weight, with respect to a total weight of the food matrix, of cultivated cells from an organism of the Animalia kingdom excluding human, of the Bacteria kingdom and/or of the Fungi kingdom, and/or cells extracts thereof. More preferably, the food matrix comprises at least 5% in weight, with respect to a total weight of the food matrix, of cultivated cells from an organism of the Animalia kingdom excluding human, of the Bacteria kingdom and/or of the Fungi kingdom, and/or cells extracts thereof. Even more preferably, the food matrix comprises at least 10% in weight, with respect to a total weight of the food matrix, of cultivated cells from an organism of the Animalia kingdom excluding human, of the Bacteria kingdom and/or of the Fungi kingdom, and/or cells extracts thereof. The weight being preferably a weight of the wet food matrix.

As it is illustrated in example, the developed technology can give good results without a large weight of cells or cells extracts. Preferably, the food matrix can comprise 99% or less in weight of cultivated cells from an organism of the Animalia kingdom excluding human, of the Bacteria kingdom and/or of the Fungi kingdom, and/or cells extracts thereof. More preferably, the food matrix comprises 95% or less in weight of cultivated cells from an organism of the Animalia kingdom excluding human, of the Bacteria kingdom and/or of the Fungi kingdom, and/or cells extracts thereof. Even more preferably, the food matrix comprises 80% or less in weight of cultivated cells from an organism of the Animalia kingdom excluding human, of the Bacteria kingdom and/or of the Fungi kingdom, and/or cells extracts thereof. The weight being preferably a weight of the wet food matrix.

The developed solution is designed to produce an edible product having appreciable meat flavor without the need to kill an animal. Preferably, the food matrix can comprise from 1% to 100% in weight of cultivated cells from an organism of the Animalia kingdom excluding human, from an organism of the Bacteria kingdom and/or from an organism of the Fungi kingdom, and/or cells extracts thereof. More preferably, the food matrix comprises from 2% to 99% in weight of cultivated cells from an organism of the Animalia kingdom excluding human, from an organism of the Bacteria kingdom and/or from an organism of the Fungi kingdom, and/or cells extracts thereof. Even more preferably, the food matrix comprises from 5% to 95% in weight of cultivated cells from an organism of the Animalia kingdom excluding human, from an organism of the Bacteria kingdom and/or from an organism of the Fungi kingdom, and/or cells extracts thereof. The weight being preferably a weight of the wet food matrix.

### Animalia Kingdom

Obviously, a solution according to the invention does not comprise the use of human cells. Moreover, a solution according to the invention preferably does not require the need to inflict suffering on animals, for example for the recovery of cells to be cultured. Regarding the Animalia kingdom, preferred cells or cells extracts for a solution according to the invention can be selected from: cultivated Mammalia cells excluding human, cultivated Aves cells, cultivated Actinopterygii cells, cultivated Malacostraca cells and/or cells extracts thereof. For example, cultivated Mammalia cells can be cultivated Bovidae cells, cultivated Cervidae cells, cultivated Leporidae cells or cultivated Suidae cells; cultivated Aves cells can be cultivated Anatidae cells or cultivated Phasianidae cells; cultivated Actinopterygii cells can be cultivated Gadidae cells, cultivated Merlucciidae cells, cultivated Pleuronectidae cells, cultivated Salmonidae cells, or cultivated Scombridae cells; and cultivated Malacostraca cells can be cultivated Palaemonidae cells.

More preferably, preferred cells or any cells extracts thereof for a solution according to the invention can be selected from: bovine cells such as cow cells or bison cells; ovine cells; galliformes cells such as chicken cells; Anatidae cells such as duck cells or goose cells; Suidae cells such as pork cells; rabbit cells; Salmonidae cells, Acipenseridae cells, Scombridae cells and/or any extracts thereof.

In particular, the food matrix can comprise at least 1% in weight (compared to wet weight of the food matrix) of cells or cells extracts selected from: cultivated Mammalia cells excluding human, cultivated Aves cells, cultivated Actinopterygii cells, cultivated Malacostraca cells and combination thereof. In particular, the food matrix can comprise at least 1% in weight (compared to wet weight of the food matrix) of cells or cells extracts selected from: Bovidae cells, Cervidae cells, Leporidae cells, Suidae cells, Anatidae cells, Phasianidae cells, Gadidae cells, Merlucciidae cells, Pleuronectidae cells, Salmonidae cells, Scombridae cells, Palaemonidae cells, and combination thereof. Preferably, the food matrix comprises at least 2%, more preferably at least 5%, even more preferably at least 10% in weight (compared to wet weight of the food matrix) of cells and/or cells extracts selected from: Bovidae cells, Cervidae cells, Leporidae cells, Suidae cells, Anatidae cells, Phasianidae cells, Gadidae cells, Merlucciidae cells, Pleuronectidae cells, Salmonidae cells, Scombridae cells, Palaemonidae cells, and combination thereof.

The cells from an organism of the Animalia kingdom excluding human can be selected from differentiated or undifferentiated cells.

The animal cells can be selected among stem cells such as embryonic stem cells, mesenchymal stem/stromal cells, induced pluripotent stem cells, fibro-adipogenic progenitors; fibroblasts; adipocytes; muscle cells such as skeletal muscle cells, cardiac cells, smooth muscle cells; chondrocytes; hepatocytes; keratinocytes; and combination thereof. Stem cells can refer to pluripotent stem cells, multipotent or totipotent cells, or to oligopotent stem cells of neurectoderm, mesoderm or endoderm lineage. Preferably, the animal cells are selected among cells derived from differentiation of non-human embryonic stem cells; cells derived from differentiation of non-human induced pluripotent stem cells; cells derived from transdifferentiated non-human isolated cells; immortalized mature non-human cells; and differentiated cells derived from differentiation of non-human progenitor cells.

For example, the food matrix can comprise at least 1% in weight of hepatocytes, preferably at least 2% in weight of hepatocytes, more preferably at least 5% in weight of hepatocytes, even more preferably at least 10% in weight of hepatocytes with respect to the total wet weight of the food matrix. Preferably, the food matrix comprises 99% or less in weight of hepatocytes, more preferably 95% or less in weight of hepatocytes, even more preferably 90% or less in weight of hepatocytes with respect to the total wet weight of the food matrix. For example, the food matrix comprises from 1% to 100% in weight of hepatocytes, preferably from 1% to 99% in weight of hepatocytes, more preferably from 2% to 95% in weight of hepatocytes, even more preferably from 5% to 90% in weight of hepatocytes with respect to the total wet weight of the food matrix. As it will be described hereafter, hepatocytes are preferably cultivated hepatocytes excluding human hepatocytes. However, in an embodiment, hepatocytes can be fresh liver hepatocytes, excluding human hepatocytes.

For example, the food matrix can comprise at least 1% in weight of myocytes (such as such as skeletal muscle cells, cardiac cells, smooth muscle cells), preferably at least 2% in weight of myocytes, more preferably at least 5% in weight of myocytes, even more preferably at least 10% in weight of myocytes with respect to the total wet weight of the food matrix. Preferably, the food matrix comprises 99% or less in weight of myocytes, more preferably 95% or less in weight of myocytes, even more preferably 90% or less in weight of myocytes with respect to the total wet weight of the food matrix. For example, the food matrix comprises from 1% to 100% in weight of myocytes, preferably from 1% to 99% in weight of myocytes, more preferably from 2% to 95% in weight of myocytes, even more preferably from 5% to 90% in weight of myocytes with respect to the total wet weight of the food matrix.

For example, the food matrix can comprise at least 1% in weight of fibroblasts, preferably at least 2% in weight of fibroblasts, more preferably at least 5% in weight of fibroblasts, even more preferably at least 10% in weight of fibroblasts with respect to the total wet weight of the food matrix. Preferably, the food matrix comprises 99% or less in weight of fibroblasts, more preferably 95% or less in weight of fibroblasts, even more preferably 90% or less in weight of fibroblasts with respect to the total wet weight of the food matrix. For example, the food matrix comprises from 1% to 100% in weight of fibroblasts, preferably from 1% to 99% in weight of fibroblasts, more preferably from 2% to 95% in weight of fibroblasts, even more preferably from 5% to 90% in weight of fibroblasts with respect to the total wet weight of the food matrix.

### Bacteria Kingdom

Regarding the Bacteria kingdom, preferred cells or cells extracts for a solution according to the invention can be selected from species belonging to Alphaproteobacteria Betaproteobacteria, Actinomycetota or Gammaproteobacteria and cells extracts thereof.

More preferably, preferred cells and/or cells extracts for a solution according to the invention can be selected from species belonging to: Pseudomonas, Bacillus, Cupriavidus, Ralstonia, Rhodococcus, Cupriavidus, Alcaligenes, Hydrogenovibrio, Rhodopseudomonas, Hydrogenobacter, Gordonia, Methylophilus, Arthrobacter, Streptomycetes, Rhodobacter, and/or Xanthobacter.

In particular, the food matrix can comprise at least 1% in weight (compared to wet weight of the food matrix) of bacterial cells and/or bacterial cells extracts thereof. Preferably, the food matrix comprises at least 2%, more preferably at least 5%, even more preferably at least 10% in weight (compared to wet weight of the food matrix) of bacterial cells and/or bacterial cells extracts thereof.

### Fungi Kingdom

Regarding the Fungi kingdom, preferred cells or cells extracts for a solution according to the invention can be selected from: Basidiomycota cells, in particular Agaricales cells or Boletaceae cells; Ascomycota cells, in particular Morchellaceae cells or Saccharomycesceae cells and/or cells extracts thereof.

More preferably, preferred cells or cells extracts for a solution according to the invention can be selected from species belonging to: Fusarium, Pleurotus, Aspergillus, Morchella, and/or Lentinula.

In particular, the food matrix can comprise at least 1% in weight (compared to wet weight of the food matrix) of fungi cells and/or fungi cells extracts thereof. Preferably, the food matrix comprises at least 2%, more preferably at least 5%, even more preferably at least 10% in weight (compared to wet weight of the food matrix) of fungi cells and/or fungi cells extracts thereof.

### Cultivated Cells

The problem of lack of flavor can be prevalent for meat like products, especially plant-based ones. Flavor of cultured cells-based products can be improved as well. The invention is particularly adapted to edible products made at least partially from cultured cells. Hence, preferably, the food matrix comprises cultivated cells or cultivated cells extracts, said cultivated cells are cells from an organism of the Animalia kingdom excluding human, cells from an of the Bacteria kingdom and/or cells from an of the Fungi kingdom, preferably said cultivated cells being cells from an organism of the Animalia kingdom excluding human.

Hence, preferably, the edible product having meat-like flavor is obtained from cultivated cells. Such cultivated cells may have been kept intact or they may have been disrupted for example through mixing, blending or homogenizing. When the cultivated cells have been disrupted, the method according to the invention can comprise a step of extracting specific compounds after the disruption. For example, the method according to the invention can comprise a step of extracting the proteins and/or lipids (such as fatty acids or phospholipids).

The food matrix can comprise animal cells excluding human cells, selected from: cells derived from differentiation of non-human embryonic stem cells; cells derived from differentiation of non-human induced pluripotent stem cells; cells derived from transdifferentiated non-human isolated cells; immortalized mature non-human cells; and differentiated cells derived from differentiation of non-human progenitor cells.

The food matrix can comprise hepatocytes excluding human hepatocytes, selected from: hepatocytes derived from differentiation of non-human embryonic stem cells; hepatocytes derived from differentiation of non-human induced pluripotent stem cells; hepatocytes derived from transdifferentiated non-human isolated cells; immortalized mature non-human hepatocytes; and differentiated hepatocytes derived from differentiation of non-human progenitor cells.

In particular, hepatocytes can be non-steatotic hepatocytes or steatotic hepatocytes.

The food matrix can comprise myocytes excluding human myocytes, for example selected from: myocytes derived from differentiation of non-human embryonic stem cells; myocytes derived from differentiation of non-human induced pluripotent stem cells; myocytes derived from transdifferentiated non-human isolated cells; immortalized mature non-human myocytes; and differentiated myocytes derived from differentiation of non-human progenitor cells.

### Differentiation of Non-Human Embryonic Stem Cells

Cells in the food matrix may be derived from differentiation of non-human embryonic stem cells. The differentiation comprises the sum of the processes whereby undifferentiated or unspecialized cells attain their function. Stem cells can be isolated from embryo and cultured using cultured media in order to achieve cellular proliferation and maintenance of the de-differentiated state. In an embodiment, the media formulations utilize synthetic serum-free media.

Next, the embryonic stem cells can be induced to differentiate for example into hepatocytes, fibroblasts, keratinocytes, myocytes or adipocytes. For example, for hepatocytes, non-human embryonic stem cells are induced into cells of the definitive endoderm, preferably with specific growth factors such as Activin A, WNT, FGF, or BMP; or other components having an effect on differentiation such as Insulin transferrin selenium, Rapamycin, KOSR, or Sodium butyrate. Next, cells of the final endoderm are specified into hepatic endoderm cells then hepatoblasts, preferably with specific factors as HGF, FGF, FGF and BMP. Hepatoblasts are differentiated into hepatocytes by differentiation induced with a combination of factors, preferably such as HGF, Oncostatin M, Dexamethasone and TGF-β. Next, the differentiated hepatocytes can be cultured and expanded to a desired quantity of cells.

### Differentiation of Non-Human Induced Pluripotent Stem Cells.

Cells in the food matrix may be derived from differentiation of non-human induced pluripotent stem cells.

An episomal reprogramming strategy, for example of avian dermal fibroblasts isolated from goose, duck or chicken, can be employed to create induced pluripotent stem cells from the fibroblasts without the use of classic viral reprogramming techniques.

The induced pluripotent stem cells can be cultured using optimized media substrates and media formulations to achieve persistent cellular proliferation and maintenance of the de-differentiated state. The media formulations utilize synthetic serum-free media. Preferably, the cells are cultured in a pathogen-free cell culture system. Next, the pluripotent stem cells can be triggered to differentiate into hepatocytes and expanded to a desired quantity of cells.

### Transdifferentiated Non-Human Isolated Cells.

Cells in the food matrix may be derived from transdifferentiated non-human isolated cells. The transdifferentiation refers to the differentiation from one differentiated cell type to another differentiated cell type, preferably, in one step. The transdifferentiation can relates to a method that modifies the differentiated phenotype or developmental potential of a cell without the formation of a pluripotent intermediate cell; i.e. it does not require that the cell be first dedifferentiated (or reprogrammed) and then differentiated to another cell type. Instead, the cell type is merely “switched” from one cell type to another without going through a less differentiated phenotype. The transdifferentiation may also comprise a first step of submitting first cells having a first cell fate to conditions to generate second cells (i.e., a less differentiated cells) that are capable of differentiating into a second cell fate; and a second step of submitting the less differentiated cells to conditions to differentiate the cells into cells having the second cell fate, such as hepatocyte cells.

For example, non-human cells such as embryonic fibroblasts, embryonic stem cells, muscle cells are isolated using techniques known in the field of cell biology and are cultivated in a media comprising basal media, antibiotics, non-essential amino acids, reducing agents, serum, minerals and growth factors.

### Immortalized Mature Non-Human Hepatocytes.

Cells in the food matrix may be selected from immortalized mature non-human hepatocytes. The specificity of the immortalized mature non-human hepatocytes lies in the fact that cells are able to divide indefinitely. Mature avian hepatocytes can be isolated from duck, goose or chicken liver. The hepatocytes can be immortalized using classical techniques such as transformation or spontaneous hepatocyte immortalization by sequentially passaging the hepatocytes until spontaneous mutations arise that result in immortalization. The immortalized hepatocytes can be expanded to a desired quantity of cells and grown in culture media.

Differentiated Cells Derived from Differentiation of Non-Human Progenitor Cells.

Cells in the food matrix may be selected from differentiated hepatocytes derived from differentiation of non-human progenitor cells. The progenitor cells can be grown using optimized media substrates and media formulations in order to obtain a persistent cellular proliferation and maintenance of the pluripotent state. The media formulations may comprise synthetic serum-free media. Then, the hepatic stem cells are induced to differentiate into mature hepatocytes and expanded to a desired quantity of cells thanks to differentiation factors.

Advantageously, regardless of the origin of the hepatocytes, the cultivated hepatocytes can be considered as cells grown in a culture medium. Advantageously, the culture medium does not comprise fetal bovine serum. The culture medium may be supplemented, preferably gradually, with hydrolysates of plant or yeast. Such serum-free media allows the reduction and elimination of animal derived components.

Various media formulations are optionally used to enable the maintenance of the capacity for self-renewal such as during expansion of the cell population. As explained, the media formulations may be modified from conventional media to not require fetal bovine serum or animal alternatives to bovine serum. Rather, the medium may include plant or yeast hydrolysates. Examples of plant-based formulations include soybean-based and plant hydrolysate-based media formulations. Some media formulations may comprise at least one ingredient for enhancing the nutritional content of the cultured cells.

In addition, the media comprises all the components and nutrients for the development of cells such as salt, glucose, water, salt minerals, and amino acids.

In an embodiment, the culture media may comprise scaffolds.

The cells are cultivated in incubators set up at 37° C., 5% of CO₂, at a pH of 7 and with moisture of at least 95%.

According to an embodiment of the invention the cultivated hepatocytes may be steatotic hepatocytes. Thus, the invention may comprise a step of inducing steatosis in said cultivated hepatocytes.

According to an embodiment, the media may be supplemented with an increased concentration of free fatty acids causing the hepatocytes to undergo steatosis by taking in and storing an excess amount of extracellular fatty acids.

Fatty acids may be selected from butyric acid, isobutyric acid, isovaleric acid, caproic acid, caprylic acid, pelargonic acid, capric acid, lauric acid, myristic acid, palmitic acid, oleic acid, docosahexaenoic acid, stearic acid, arachidic acid, linoleic acid, linolenic acid, arachidonic acid, palmitoleic acid and eicosapentaenoic acid or a mixture of thereof.

The length of time during which the cells are exposed to lipid concentrations sufficient to induce steatosis will vary depending on the cell type, size of the cell population, age of the cell population, number of passages, any genetic modification or manipulation of the cells, the type and components of the culture media, desired amount of lipid accumulation or steatosis, or any combination thereof.

For example, certain cell types will intake exogenous lipids from the culture media at a slower rate than other cell types, and thus require a longer incubation period in a lipid rich media to induce the desired amount of steatosis.

In many cases, cells are cultured in a culture media having a high lipid concentration for about 30 days to about 60 days.

According to another embodiment, the steatosis may be induced by the increased expression in the cell of the lipid pathway responsible for lipid metabolism and storage. In this case, the genetic manipulation results in the accumulation of lipid droplets within the cytoplasm of the hepatocytes, thereby resulting in steatosis. Expression of genes involved in lipid metabolism can be induced or enhanced to facilitate or enhance lipid accumulation and/or steatosis in target cells such as hepatocytes. According to an embodiment of the invention the hepatocytes in which steatosis is induced may be non-steatotic hepatocytes.

As stated, the food matrix comprises cells from an organism of the Animalia kingdom excluding human, from an organism of the Bacteria kingdom and/or from an organism of the Fungi kingdom, preferably cultivated cells.

First, it should be understood for all the embodiments of the present invention that the cellular origins are not mutually exclusive. Hence, for example, if the food matrix comprises cells from an organism of the Animalia kingdom excluding human, it can also comprise cells from an organism of the Bacteria kingdom or cells from an organism of the Fungi kingdom. In another example, if the food matrix comprises cells from an organism of the Fungi kingdom, it can also comprise cells from an organism of the Bacteria kingdom and cells from an organism of the Animalia kingdom excluding human.

Second, it should be understood for all the embodiments of the present invention that the food matrix can further comprise cells from an organism which do not belong to the Animalia kingdom excluding human, of the Bacteria kingdom or the Fungi kingdom. For example, the food matrix can comprise plant cells or plant extracts (such as protein concentrate, protein isolate and/or plant fat). In particular, the food matrix further comprises at least 1% in weight of plant cells.

Third, it should be understood for all the present invention that the food matrix can comprise compounds in addition to the cells. For example, the food matrix can comprise fatty acids and/or phospholipids.

For example, the food matrix can comprise plant fat, such as fatty acids, which may come from plant oils such as canola seed (rapeseed), castor, coconut, flaxseed, allanblackia, olive, sunflower, soybean, peanut, illipe, cottonseed, shea, palm, avocado, safflower, sesame, lemon, grapeseed, macadamia, almond, sal, kokum, mango or a combination thereof. Preferably, the food matrix comprises a mixture of fat from different plants.

Hence a method according to the invention can comprise, prior to the step of treating 140 the food matrix, a homogenization of cultivated cells and/or cells extracts thereof with fat. Preferably, said fat being a non-human animal fat, a plant-based fat, a fermented fat or a mixture thereof. As this fat can be added before the step of treating 140 the food matrix, this mixture is, at the time of homogenization or subsequently, treated with a ROS producing enzyme to produce the treated food matrix.

The food matrix can comprise at least 1% of lipids in weight compared to total food matrix weight, preferably at least 2% of lipids in weight, more preferably at least 5% of lipids in weight, even more preferably at least 10% of lipids in weight, for example at least 20% of lipids in weight, compared to total food matrix weight (preferably compared to total food matrix wet weight).

Preferably the fat used in a method according to the invention comprises mainly triglycerides. Hence, the food matrix can comprise at least 1% in weight of triglycerides compared to total food matrix weight, preferably at least 2% of triglycerides in weight, more preferably at least 5% of triglycerides in weight, even more preferably at least 10% of triglycerides in weight compared to total food matrix weight. Preferably, the triglycerides comprise more oleic acids than palmitic acids in weight.

Moreover, the fat used in a method according to the invention comprises a high concentration of unsaturated fatty acids. Hence, the food matrix can comprise at least 0.5% of unsaturated fatty acids in weight compared to total food matrix wet weight, preferably at least 1% of unsaturated fatty acids in weight, more preferably at least 2% of unsaturated fatty acids in weight, even more preferably at least 3% of unsaturated fatty acids in weight compared to total food matrix wet weight. Such unsaturated fatty acids are preferably polyunsaturated fatty acids (PUFA). Hence, the food matrix comprises at least 0.1% of PUFA in weight compared to total food matrix weight, preferably at least 0.2% of PUFA in weight, more preferably at least 0.5% of PUFA in weight, even more preferably at least 1% of PUFA in weight compared to total food matrix wet weight. Moreover, in a preferred embodiment, these PUFA are long chain unsaturated fatty acids. Hence, the food matrix comprises at least 0.1% of PUFA>C18 compared to total food matrix weight, preferably at least 0.2% of PUFA>C18 in weight, more preferably at least 0.3% of PUFA>C18 in weight, even more preferably at least 0.5% of PUFA>C18 in weight compared to total food matrix weight. PUFA>C18 refers to polyunsaturated fatty acids having more than 18 carbon atoms, such as C20 PUFA or C22 PUFA.

The concentration of lipids, in particular polyunsaturated fatty acids, in the composition relating to the invention has an impact on the meat-like flavor.

A method 100 of producing an edible product having meat-like flavor according to the invention can comprise a step of adding a food additive 120. This step is in particular designed to improve the flavor or the texture of the edible product. The edible product may comprise between 0.01 and 10% in weight of food additive, preferably between 0.01 and 4% in weight of food additive with respect to the total wet weight of the edible product.

As illustrated in FIG. 1, a method according to the invention can comprise a food additive addition before 121 and/or after 122, 123 the step of treating 140 the food matrix.

The food additive can be selected from seasoning, flavoring additive, texturizer additive, food colorant, preservative additive or a combination thereof.

A seasoning can for example be selected from: salt; pepper; aromatic herbs and/or spices, including rosemary, sage, mint, oregano, parsley, thyme, bay leaf, cloves, basil, chives, marjoram, nutmeg, cardamom, chiles, cinnamon, fennel, fenugreek, ginger, saffron, vanilla and coriander; alcohol, including wine, spirituous, cognac, armagnac; or any combination thereof.

A flavoring additive can for example be selected from: flavor enhancer, sweetener or any combination thereof.

A texturizer additive can for example be selected from: bulking agent or thickener, desiccant, curing agent or any combination thereof,

A preservative additive can for example be selected from: antimicrobial agent, pH modulator, or any combination thereof.

A food colorant can for example be selected from natural colorants such as carotenes, tomato, beet, or a mixture thereof. The edible product may comprise between 0.01 and 4% of food colorants.

As shown in FIG. 1, a method 100 of producing an edible product having meat-like flavor according to the invention can comprise a homogenization 130 step. This step is in particular designed to homogenize the food matrix and to mix all the components together when the food matrix has been supplemented with other ingredients such as plant material or food additives.

The food matrix can be homogenized before 131 the step of treating 140 the food matrix. The food matrix can be homogenized after 132 the step of treating 140 the food matrix. During this step, an emulsion can be created. This is particularly advantageous when the food matrix has been supplemented with a fat composition.

Preferably, the solution, and preferably the fatty solution, is gradually added under stirring or alternatively, the composition is mixed with the liquid fat until homogeneity. Optionally, the solution, and preferably the fatty solution and the composition can be brought to the same temperature before mixing for example to a temperature between 30° C. and 100° C.

Preferably, the homogenization 130 step is performed with a blender, a high-speed mixer, or with a homogenizer, as a rotor-stator homogenizer, a cutter or a colloid mill.

The homogenization 130 step may be conducted during at least 10 sec, preferably at least 30 sec, more preferably at least 50 sec, even more preferably at least 100 sec, for example at least 150 sec.

The homogenization 130 step may be conducted at least 100 rpm, preferably at least 300 rpm, more preferably at least 500 rpm, even more preferably at least 1000 rpm, for example at least 2000 rpm.

The homogenization will preferably induce an emulsion, more preferably a microemulsion in the food matrix. Without being limited by the theory, such microemulsion improves the effect of the reactive oxygen species producing enzyme on the food matrix. An emulsion or microemulsion according to the present invention will generate lipidic droplets with a mean diameter of the droplet size distribution of less than 25 μm, preferably less than 20 μm, more preferably less than 15 μm and even more preferably less than 10 μm. The mean diameter of the droplet size distribution can be measured by dynamic light scattering, for example according to ISO 22412 and/or with a Mastersizer 3000 de malvern panalytical device.

As shown in FIG. 1, a method 100 of producing an edible product having meat-like flavor according to the invention comprises a step of treating 140 the food matrix with a reactive oxygen species producing enzyme.

This step is in particular designed to improve the organoleptic qualities of the edible product. In particular, such treatment improves the meat-like flavor of a food matrix. Hence, such step of treating 140 the food matrix allows to obtain a treated food matrix having meat-like flavor, preferably a meat-like flavor enhanced compared to the meat-like flavor of the untreated food matrix.

Generally, ROS are known to be highly reactive, detrimental to the integrity of proteins, lipids and carbohydrates. However, such treatment according to the invention improves flavor and more particularly meaty taste, aroma, color, meaty smell such as a smell of “foie-gras” in particular when the food matrix comprises cells from an organism of the Animalia kingdom excluding human, from an organism of the Bacteria kingdom and/or from an organism of the Fungi kingdom, and/or cells extracts thereof.

As it is described in examples, where the use of ROS induces poor results on the flavor of the treated food matrix, the use of a reactive oxygen species producing enzyme in combination with above mentioned cells, and preferably combined with an enzyme inactivation step (e.g. through heat treatment), helps to develop complex flavor associated with meat-like flavor.

Modifications of food molecules, in particular with enzymes, have already been done but it has never been proposed a method for edible product production comprising the use of reactive oxygen species producing enzyme(s) to produce an edible product having meat-like flavor, from a food matrix comprising cells from an organism of the Animalia kingdom excluding human, from an organism of the Bacteria kingdom, and/or from an organism of the Fungi kingdom, and/or cells extracts thereof, advantageously cultivated cells.

For example, the ROS producing enzyme can be an enzyme producing: peroxides such as hydrogen peroxide, superoxide, hydroxyl radicals, singlet oxygen, nitric oxide, peroxinitrite, free radicals, hypochlorite or hypochlorous acid, peroxyl radicals such as alkylperoxyl and hydroperoxyl, alkoxyl radicals, or combination thereof.

Preferably, the ROS producing enzyme can be an enzyme producing hydrogen peroxide.

In particular, the ROS producing enzyme can be selected from superoxide dismutase, glucose oxidase, catalase, glutathione peroxidase, glutathione reductase, gluconate kinase, gluconate-6-phosphate dehydrogenase, gluconolactonase, cholesterol oxidase, nitric oxide synthase, lipoxygenase, hexose oxidase, galactose oxidase, pyranose oxidase, choline oxidase, pyruvate oxidase, glycolate oxidase, amino acid oxidase, NAD (P) H oxidase, lipases or combination thereof.

Preferably, the ROS producing enzyme can be selected from superoxide dismutase, glucose oxidase, lipase or combination thereof.

More preferably, the ROS producing enzyme can be selected from superoxide dismutase or glucose oxidase or combination thereof.

It should be understood for the present invention that the use of a ROS producing enzyme to treat the food matrix can encompass the use of several ROS producing enzymes. Indeed, the invention can encompass the use of only one kind of ROS producing enzymes, but it can also encompass the use of several ROS producing enzymes or the use of several enzymes which together produce ROS.

The ROS producing enzyme can be a native enzyme or synthetic versions of the enzyme.

Superoxide dismutase catalyzes O₂.— to produce H₂O₂, and more particularly, catalyzes the dismutation of O₂·— to H₂O₂. In addition, and advantageously, the superoxide dismutase enhances steatosis.

Glucose oxidase catalyzes the conversion of glucose to hydrogen peroxide and D-glucono-δ-lactone.

The step of treating the food matrix can comprise adding a ROS-producing enzyme at a concentration of at least 0.0001 IU per gram of food matrix (wet weight), preferably at least 0.0005 IU per gram of food matrix (wet weight), more preferably at least 0.001 IU per gram of food matrix (wet weight), even more preferably at least 0.005 IU per gram of food matrix (wet weight).

The step of treating the food matrix can comprise adding an enzyme producing ROS at a concentration of at most 10 IU per gram of food matrix (wet weight), preferably at most 5 IU per gram of food matrix (wet weight), more preferably at most 2 IU per gram of food matrix (wet weight), even more preferably at most 1 IU per gram of food matrix (wet weight), for example at most 0.1 IU per gram of food matrix (wet weight).

The step of treating the food matrix can comprise adding an enzyme producing ROS at a concentration from 0.0001 IU per gram to 10 IU per gram of food matrix (wet weight), preferably from 0.0005 IU per gram to 5 IU per gram, more preferably from 0.001 IU per gram to 2 IU per gram, even more preferably from 0.005 IU per gram to 1 IU per gram of food matrix (wet weight).

Increasing the concentration of ROS producing enzymes results in a more pronounced flavor change.

For example, the ROS producing enzyme can be an enzyme producing hydrogen peroxide, and the hydrogen peroxide producing enzyme can be used at a concentration from 0.001 to 100 IU per gram of food matrix (wet weight), preferably from 0.005 to 50 IU per gram, more preferably from 0.01 to 25 IU per gram, even more preferably from 0.05 to 15 IU per gram, for example from 0.05 to 10 IU per gram.

Advantageously, when the food matrix comprises Anatidae cells (e.g. duck or goose), in particular hepatocytes, adding a ROS producing enzyme decreases the liver flavor and increases the foie gras flavor. Hence, the edible product has improved organoleptic qualities, with appetizing profile, olfactory and gustatory sensations felt during the tasting equivalent to that of a foie gras resulting from a force-feeding.

Advantageously, when the food matrix comprises Actinopterygii cells (e.g. salmon or tuna), adding a ROS producing enzyme can increase the fish flavor without generating off-flavor. Hence, the edible product has improved organoleptic qualities, with appetizing profile, olfactory and gustatory sensations felt during the tasting, equivalent to that of a fish that sourced from the sea, rivers, aquaculture or pisciculture.

Advantageously, when the food matrix comprises Bovidae cells, adding a ROS producing enzyme can increase the marbled meat flavor without generating off-flavor. Hence, the edible product has improved organoleptic qualities, with appetizing profile, olfactory and gustatory sensations felt during the tasting equivalent to that of a Bovidae that grew up in the fields.

The step of treating 140 the food matrix involves contact between the ROS producing enzyme and the food. The step of treating 140 the food matrix can comprise adding an enzyme producing ROS in the food matrix. The ROS producing enzyme can be in contact with the food matrix during at least 30 sec, preferably at least one minute, more preferably at least two minutes, even more preferably at least three minutes, before being inactivated or removed during the processing step. The ROS producing enzyme can be in contact with the food matrix during at most ten hours, preferably at most five hours, more preferably at most two hours, even more preferably at most one hour, for example at most thirty minutes before being inactivated or removed during the processing step. The ROS producing enzyme can be in contact with the food matrix during 30 sec to three hours, preferably one minute to two hours, more preferably two minutes to sixty minutes, even more preferably three minutes to thirty minutes, before being inactivated or removed during the processing step.

As it has already been described, the treating 140 step can be done during a homogenization of the food matrix. The step of treating 140 the food matrix can be performed using an homogenizer, a blender, a mixer, a cutter, a grinder, an extruder. Preferably, the step of treating 140 the food matrix can be performed in conjunction with step of homogenizing 130 if the ROS producing enzyme is in the free form or as an adjacent unit step upon entry/exit of step of homogenizing 130 if the ROS producing enzyme is in an immobilized form.

In some cases, during the treating 140 step, it will be preferable to have a controlled protein concentration in the food matrix. Hence, the food matrix can comprise at most 80% of proteins in weight compared to total food matrix weight, preferably at most 60% of proteins in weight, more preferably at most 50% of proteins in weight, even more preferably at most 40% of proteins in weight, for example at most 30% of proteins in weight, compared to total food matrix weight (preferably compared to total food matrix wet weight).

Preferably, the food matrix comprises a ratio in weight of unsaturated fat on protein weight of at least 1, preferably at least 1.25, more preferably at least 1.50, even more preferably at least 2.

As shown in FIG. 1, a method 100 of producing an edible product having meat-like flavor according to the invention can comprise a heat treatment 150 step. In particular, this step can be designed to cook or precook the edible product and to develop its flavors, in particular complex flavors. This step can also be designed to sterilize the edible product.

An advantage of this heat treatment step is that it can inactivate the ROS producing enzyme. Hence, this step can be used to inactivate the ROS producing enzymes that remain in the food matrix. Advantageously, the heat treatment step also ensures the microbial safety of the edible product according to the invention used as a meat substitute, in particular the edible product according to the invention used as a foie gras substitute. It also plays a major role in defining final product quality characteristics such as flavor, color and texture.

The heat treatment 150 step can be conducted until a core temperature of at least 40° C. is reached, preferably at least 50° C., more preferably at least 60° C. and even more preferably at least 70° C. The heat treatment 150 step can be conducted during at least ten seconds, for example at least one minutes, preferably at least five minutes, more preferably at least ten minutes, even more preferably at least fifteen minutes.

The heat treatment 150 step can be conducted until a core temperature of at most 130° C. is reached, preferably at most 100° C., more preferably at most 90° C. and even more preferably at most 80° C. The heat treatment 150 step can be conducted during at most 120 minutes, preferably at most 60 minutes, more preferably at most 30 minutes, more preferably a most 15 minutes.

The heat treatment 150 step can be conducted until a core temperature from 40° C. to 130° C. is reached, preferably from 50° C. to 90° C., more preferably from 55° C. to 85° C. and even more preferably from 60° C. to 80° C. The heat treatment 150 step can be conducted during 5 to 60 minutes, preferably 5 to 30 minutes, more preferably 5 to 15 minutes.

Alternatively, the heat treatment step can correspond to a sterilization or a pasteurization.

The heat treatment step can be done using a conveyor belt and a oven, the oven is a tunnel oven inside which the conveyor belt runs; an oven; a hot liquid bath, steam projection, microwave or radiofrequency irradiation.

According to an embodiment, the method may comprise, for example after the step of heat treatment, a cooling step. Preferably, the cooling step is performed in a water bath or a cool room for example during at least one night.

As shown in FIG. 1, a method 100 of producing an edible product having meat-like flavor according to the invention comprises a step of processing 160 the treated food matrix. Preferably, said processing the treated food matrix including an inactivation or a removal of the ROS producing enzyme.

This step is in particular designed to obtain an edible product having meat-like flavor. After the treatment of the food matrix by the ROS producing enzyme, the ROS producing enzyme should be removed from the food matrix or inactivated.

Preferably, the removal of the ROS producing enzyme can comprise the use of a ROS producing enzyme which is immobilized on a support that can be incubated with the food matrix during the enzymatic treatment before removal of the support. Hence, the treated food matrix can be flushed out and another batch of untreated food matrix can be incubated with the immobilized ROS producing enzyme.

If the enzyme is added in a free form in the food matrix, it can be inactivated after the food matrix treatment. The person skilled in the art knows several ways of inactivating the enzyme which should be adapted to the enzyme. Preferably, the inactivation includes a heat treatment to induce a denaturing temperature, a modification of the pH of the food matrix to induce a denaturing pH or a modification of the ionic strength of the food matrix to induce a denaturing ionic strength. More preferably, the inactivation of the ROS producing enzyme during the processing 160 the treated food matrix can correspond to the heat treatment 150.

Beyond this inactivation or a removal, in the food industry, there is a great variety of ways to prepare an edible product, whether it is a ready to eat edible product or an intermediate edible product such as an ingredient for the preparation of a ready to eat edible product.

According to the invention, the step of processing 160 the treated food matrix can comprise steps such as steps that affect water content, shape or texture, flavor or even shelf life.

For example, according to the invention, the step of processing 160 the treated food matrix can comprise steps such as drying, desiccation, dehydration, lyophilization, filtering or a combination thereof.

For example, according to the invention, the step of processing 160 the treated food matrix can comprise steps such as sterilization or pasteurization.

For example, according to the invention, the step of processing 160 the treated food matrix can comprise steps such as crushing, squishing, cutting, grinding, mixing, shredding, squeezing or a combination thereof. It can also comprise baking or cooking steps such as smoking, roasting, frying, surface treatment, coating or a combination thereof.

Finally, according to the invention, the step of processing 160 the treated food matrix can comprise steps such as cooling, refrigeration, deep-freezing packaging or a combination thereof.

In another aspect, the invention relates to an edible product having a meat-like flavor.

This edible product having a meat-like flavor is obtainable by a method according to the invention. Preferably, an edible product having a meat-like flavor has been obtained by a method according to the invention. Preferably, the edible product having a meat-like flavor comprises a treated food matrix according to the, whether preferred or not preferred, embodiment described earlier in the description of a method according to the invention.

Advantageously, the edible product having a meat-like flavor has been produced from cultivated cells. Hence, as it will be detailed hereafter, the edible product having meat-like flavor according to the invention can comprise intact cultivated cells, disrupted cultivated cells and/or extracts of cultivated cells (such as extracts of disrupted cultivated cells). In particular, the edible product comprises a treated food matrix comprising cultivated cells (i.e. intact or disrupted) or cultivated cells extracts, said cells being cultivated cells from an organism of the Animalia kingdom excluding human, from an organism of the Bacteria kingdom and/or from an organism of the Fungi kingdom.

The edible product can comprise at least 0.5% in weight, with respect to a total weight of the edible product, of cultivated cells from an organism of the Animalia kingdom excluding human, cultivated cells from an organism of the Bacteria kingdom and/or cultivated cells from an organism of the Fungi kingdom, and/or cells extracts thereof. Preferably, the edible product comprises at least 1% in weight, with respect to a total weight of the edible product, of cultivated cells from an organism of the Animalia kingdom excluding human, of the Bacteria kingdom and/or of the Fungi kingdom, and/or cells extracts thereof. More preferably, the edible product comprises at least 2% in weight, with respect to a total weight of the edible product, of cultivated cells from an organism of the Animalia kingdom excluding human, of the Bacteria kingdom and/or of the Fungi kingdom, and/or cells extracts thereof. Even more preferably, the edible product comprises at least 5% edible product, with respect to a total weight of the edible product, of cultivated cells from an organism of the Animalia kingdom excluding human, of the Bacteria kingdom and/or of the Fungi kingdom, and/or cells extracts thereof. The weight being preferably a weight of the wet edible product. Preferably, the percentage refers to cultivated cells or cultivated cells extract from an organism of the Animalia kingdom excluding human.

As it is illustrated in example, the developed technology can give good results without a large weight of cells or cells extracts. The edible product can comprise 70% or less in weight of cultivated cells from an organism of the Animalia kingdom excluding human, of the Bacteria kingdom and/or of the Fungi kingdom, and/or cells extracts thereof. For example, the edible product can comprise 50% or less in weight of cultivated cells from an organism of the Animalia kingdom excluding human, of the Bacteria kingdom and/or of the Fungi kingdom, and/or cells extracts thereof. Preferably, the edible product comprises 40% or less in weight of cultivated cells from an organism of the Animalia kingdom excluding human, of the Bacteria kingdom and/or of the Fungi kingdom, and/or cells extracts thereof. More preferably, the edible product comprises 30% or less in weight of cultivated cells from an organism of the Animalia kingdom excluding human, of the Bacteria kingdom and/or of the Fungi kingdom, and/or cells extracts thereof. Even more preferably, the edible product comprises 20% or less in weight of cultivated cells from an organism of the Animalia kingdom excluding human, of the Bacteria kingdom and/or of the Fungi kingdom, and/or cells extracts thereof. The weight being preferably a weight of the wet edible product. Preferably, the percentage refers to cultivated cells or cultivated cells extract from an organism of the Animalia kingdom excluding human.

The developed solution is designed to produce an edible product having appreciable meat flavor without the need to kill an animal. The edible product can comprise from 0.5% to 70% in weight of cultivated cells from an organism of the Animalia kingdom excluding human, from an organism of the Bacteria kingdom and/or from an organism of the Fungi kingdom, and/or cells extracts thereof. For example, the edible product can comprise from 0.5% to 50% in weight of cultivated cells from an organism of the Animalia kingdom excluding human, from an organism of the Bacteria kingdom and/or from an organism of the Fungi kingdom, and/or cells extracts thereof. Preferably, the edible product can comprise from 1% to 40% in weight of cultivated cells from an organism of the Animalia kingdom excluding human, from an organism of the Bacteria kingdom and/or from an organism of the Fungi kingdom, and/or cells extracts thereof. More preferably, the edible product comprises from 2% to 30% in weight of cultivated cells from an organism of the Animalia kingdom excluding human, from an organism of the Bacteria kingdom and/or from an organism of the Fungi kingdom, and/or cells extracts thereof. Even more preferably, the edible product comprises from 5% to 20% in weight of cultivated cells from an organism of the Animalia kingdom excluding human, from an organism of the Bacteria kingdom and/or from an organism of the Fungi kingdom, and/or cells extracts thereof. The weight being preferably a weight of the wet edible product. Preferably, the percentage refers to cultivated cells or cultivated cells extract from an organism of the Animalia kingdom excluding human.

Moreover, the edible product having a meat-like flavor can comprise a reactive oxygen species (ROS) producing enzyme. Preferably, a ROS producing enzyme which has been inactivated. For example, an edible product according to the invention comprises a ROS producing enzyme at a concentration of at least 0.00001 ng/g of edible product (wet weight), preferably at least 0.0001 ng/g of edible product (wet weight), more preferably at least 0.001 ng/g of edible product (wet weight), even more preferably at least 0.01 ng/g of edible product (wet weight). The ROS producing enzyme concentration can be measured for example by mass spectrometry.

As mentioned, the fat used in an edible product according to the invention comprises preferably a high concentration of unsaturated fatty acids. Hence, the edible product can comprise at least 0.1% of unsaturated fatty acids in weight compared to total edible product wet weight, preferably at least 0.5% of unsaturated fatty acids in weight, more preferably at least 1% of unsaturated fatty acids in weight, even more preferably at least 2% of unsaturated fatty acids in weight compared to total food matrix wet weight. Such unsaturated fatty acids are preferably polyunsaturated fatty acids (PUFA). Hence, the food matrix comprises at least 0.05% of PUFA in weight compared to total food matrix weight, preferably at least 0.1% of PUFA in weight, more preferably at least 0.25% of PUFA in weight, even more preferably at least 0.5% of PUFA in weight compared to total food matrix wet weight. Moreover, in a preferred embodiment, these PUFA are long chain unsaturated fatty acids. Hence, the food matrix comprises at least 0.05% of PUFA>C18 compared to total food matrix weight, preferably at least 0.10% of PUFA>C18 in weight, more preferably at least 0.15% of PUFA>C18 in weight, even more preferably at least 0.20% of PUFA>C18 in weight compared to total food matrix weight. PUFA>C18 refers to polyunsaturated fatty acids having more than 18 carbon atoms, such as C20 PUFA or C22 PUFA.

The edible product according to the invention is preferably easy to cut, handle, transport or spread. It can take a liquid form, such as a suspension or a solution, a solid form, such as a powder, or semi-solid form such as a paste form. Preferably, the edible product comprises the treated food matrix as an emulsion, preferably a microemulsion.

In particular, the invention can concern an edible product having meat-like flavor and in particular a foie gras flavor, without hepatocytes obtained from a force-fed animal. Preferably, the edible product with foie gras flavor is obtainable by a method according to the invention.

Preferably, an edible product having a meat-like flavor according to the invention comprises at least 51% in weight of lipids with respect to the total wet weight of the edible product. More preferably, an edible product having a meat-like flavor according to the invention comprises at least 55% in weight of lipids with respect to the total wet weight of the edible product. Even more preferably, an edible product having a meat-like flavor according to the invention comprises at least 60% in weight of lipids with respect to the total wet weight of the edible product.

### EXAMPLES

The invention is further described in detail by reference to the following experimental examples. These examples are provided for purposes of illustration only and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the compounds of the present invention and practice the claimed methods. The following examples, therefore, specifically point out the preferred embodiments of the present invention, and are not to be construed as limiting in any way the remainder of the disclosure.

As it has been detailed, the invention comprises the use of cultivated cells for producing an edible product having meat-like flavor. These examples are in particular directed to such an aspect.

### Sensory Evaluation and Study Design

The sensory evaluations include more than 8 panelists trained for more than 20 hours prior to the sensory trial to calibrate the panel with the defined attributes. Products are presented using a controlled random sampling order, a uniform sample size, and a controlled temperature to help reduce bias. Additionally, panelists are offered tap water for rinsing between samples and a cracker, to reset flavor receptors.

Triangle difference tests (ASTM E1885) were conducted for an edible product preparation to determine if a perceptible difference exists between a ROS treatment and the type of ROS treatment (enzyme or ROS) versus the traditional meat product. Furthermore, one of the goals of the triangle difference tests can be to determine the similarities between edible products according to the invention and their traditional meat counterparts in the case of no detected difference.

Moreover, panelists evaluate the edible product samples before and after tasting the products on overall liking and meat-like flavor. For example, when foie gras substitutes are tested, panelists evaluate the edible product samples also on foie gras flavor.

### Example 1: Edible Product with Meat-Like Flavor from Duck Cells

### Food Matrix Preparation

Duck cells were duck cells obtained from biopsies or cultivated cells. Cultivated cells can be duck embryonic stem cells first cultured to achieve persistent cellular proliferation and maintenance of an undifferentiated state. The embryonic stem cells are induced to differentiate into definitive endoderm cells and further mature hepatocytes using HGF and bFGF cytokines. The mature hepatocytes are then harvested and processed by centrifugation to remove the culture medium, optionally at least one washing/centrifugation cycle is applied on cells to remove culture medium. The mature hepatocytes are then considered as duck cells suitable for use in a food matrix.

The duck cells were mixed in a beaker for 10 minutes with plant fat and various food additives such as salt, peppers and fructose. Two master preparations were made with two duck cells to plant fat ratio.

For each master preparations, several times 50 grams was weighted:
for a non-treated sample (1A & 2A),
for a sample treated (1B & 2B) with ROS (hydrogen peroxide), and
for a treated sample (1C & 2C) with 20 μl of 25 000 IU/g of a ROS producing enzyme.

The Table 2 below shows the quantity used for each of the preparations. The preparation 1A, 1B, 2A, 2B are comparative (comp.) examples. They are not related to the invention.

TABLE 2

| | 1A | 1B | 1C | 2A | 2B | 2C |
|---|---|---|---|---|---|---|
| Ingredients | (comp.) | (comp.) | (invention) | (comp.) | (comp.) | (invention) |
| Plant Fat (% w) | 19.2 | 19.2 | 19.2 | 67.2 | 67.2 | 67.2 |
| Duck cells (% w) | 78 | 78 | 78 | 30 | 30 | 30 |
| Salt | 1 | 1 | 1 | 1 | 1 | 1 |
| D-Fructose | 1.5 | 1.5 | 1.5 | 1.5 | 1.5 | 1.5 |
| White Pepper | 0.3 | 0.3 | 0.3 | 0.3 | 0.3 | 0.3 |
| Total(% w) | 100 | 100 | 100 | 100 | 100 | 100 |
| ROS producing | 0 | 0 | 20 | 0 | 0 | 20 |
| enzyme (μl) | | | | | | |
| ROS - H₂O₂ (μl/g) | 0 | 15 | 0 | 0 | 15 | 0 |

### Food Matrix Treatment

The duck cells with the seasonings (salt, pepper and fructose) were mixed using an industry standard high-shear mixing or a dispersion technology at 5000 RPM, 15° C., for about two minutes.

Then the plant fat is added, and the mixture is mixed again using an industry standard high-shear mixing or a dispersion technology at 5000 RPM, 15° C. for about two minutes.

Depending on the preparation, the ROS (1B & 2B) or the ROS producing enzyme (1C & 2C) is added and the preparation is mixed during ten minutes at 5000 RPM, 15° C.

The preparations were poured in a jar and then cooked at 70° C. (water bath) for 5-10 min and cooled down before storing it in the fridge.

### Sensory Evaluation Results

For the sensory analysis, samples were served in groups of three such as:
Two of the negative control (e.g. 1A), and one of the samples of treated with the enzyme (e.g. 1C) or ROS (e.g. 1B);
Two of the samples treated with the enzyme (e.g. 2C) or ROS (e.g. 2B), and one of the negative control (e.g. 2A);
Two of the sample of interest treated with the enzyme (e.g. 2C), and one of the sample treated with the ROS (e.g. 2B); or
Two of the samples treated with the ROS (e.g. 2B) and one of the samples of interest treated with the enzyme (e.g. 2C).

Food samples were anonymized before tasting in which a triangle test discrimination was conducted to determine the odd sample.

Results of the triangle tests for flavor indicated that panelists could distinguish changes induced by the addition of ROS or by the addition of a ROS producing enzyme.

Panelists were also able to detect differences in ROS treated samples and samples treated by the ROS producing enzyme. These results were consistent with the results of the descriptive analysis. Indeed, the descriptive analysis suggest while the addition of ROS induce the generation of off-odors and off-flavors such as rancid odor, the addition of a ROS producing enzyme induce the generation of complex flavor, enhancing the meat-like flavor of the product compared to untreated sample.

Furthermore, an increase of the percentage of fat treated with the ROS producing enzyme (2C) can induce a more pronounced and more complex meat-like flavor.

### Example 2: Edible Product with Meat-Like Flavor from Other Cells

### Food Matrix Preparation

Fish cells or Beef cells are cells obtained from biopsies or cultivated cells. Cultivated cells can be embryonic stem cells first cultured to achieve persistent cellular proliferation and maintenance of an undifferentiated state. Cultivated cells can also be differentiated cells such as bovine muscle cells, or fish muscle cells.

Cells are seeded in standard medium (e.g. DMEM+10 percent Fetal Bovine Serum+1 percent penicillin\streptomycin). 24 hours after seeding the cells are seeded in a bioreactor and cultivated in their respective medium for five days. The cells biomass is then harvested and processed by centrifugation for 10 minutes at 1,500 g (at 4 degrees C.) to remove cell debris and the culture medium, optionally at least one washing/centrifugation cycle is applied on cells to remove culture medium. The cell biomass is then considered as cultivated cells suitable for use in a food matrix.

The cultivated cells are mixed in a beaker for 10 minutes with various food additives such salt, pepper and fructose. For preparation, two times 50 grams is weighted:
One for a non-treated sample, or
One for a treated sample with 20 μl of 25 000 IU/g of a ROS producing enzyme.

A comparative example is also made from soybean treated or not treated with ROS producing enzyme. 50 grams of ground soybeans with the seasonings (salt, pepper and fructose) is weighted and treated or not with 20 μl of 25 000 IU/g of a ROS producing enzyme.

### Food Matrix Treatment

The cultivated cells with the seasonings (salt, pepper and fructose) or soybeans are mixed with an industry standard high-shear mixing or a dispersion technology at 5000 RPM, 15° C. for about two minutes.

Eventually a plant fat is added, and the mixture is mixed with an industry standard high-shear mixing or a dispersion technology at 5000 RPM, 15° C. for about two minutes.

For the treated samples, the ROS producing enzyme is added and the preparation is mixed for ten minutes at 5000 RPM, at room temperature.

The preparations are poured in a jar and then cooked at 70° C. (water bath) for 5-10 min and cooled down before storing it in the fridge.

### Sensory Evaluation Results

Food samples are anonymized before tasting in which a triangle test discrimination was conducted to determine the odd sample. The table 3 below illustrates results of a triangle test discrimination.

TABLE 3

| | |
|---|---|
| Salmon cells | enhancement positive |
| Haddock cells | enhancement positive |
| Beef cells | enhancement positive |
| Ground soybean | negative |

The panelists can distinguish changes and generation of complex flavors associated with a meat-like flavor induced by the addition of a ROS producing enzyme. The addition of a ROS producing enzyme induces the generation of complex flavor, enhancing the meat-like flavor of the product compared to the untreated sample. This is not the case for the ground soybean sample.

While the meat industry is typically looking for ways to minimize lipid oxidation in order to prevent the generation of unwanted molecules, the invention shows that an enzymatic mediated oxidation can enhance the taste of meat. Hence, the ROS-mediated flavor generation by ROS producing enzymes can be a useful asset for producing more complex meat flavor profiles, in particular in products comprising cultivated cells that can require flavor enrichment. Instead of adding numerous flavor additives, a ROS producing enzyme can induce a range of chemical reactions to generate a variety of aroma compounds, while preventing uncontrollable oxidation by inactivating the ROS producing enzyme in time.

The invention can be the subject of numerous variants and applications other than those described above. In particular, unless otherwise indicated, the different structural and functional characteristics of each of the implementations described above should not be considered as combined and/or closely and/or inextricably linked to each other, but on the contrary as simple juxtapositions. In addition, the structural and/or functional characteristics of the various embodiments described above may be the subject in whole or in part of any different juxtaposition or any different combination.

1. Method of producing, from cultivated cells, an edible product having a meat-like flavor, the method comprising the steps of:
Providing a food matrix, said food matrix comprising cultivated cells and/or extracts thereof, said cultivated cells comprising cells from an organism of the Animalia kingdom excluding human, cells from an organism of the Bacteria kingdom and/or cells from an organism of the Fungi kingdom;
Treating the food matrix with a reactive oxygen species (ROS) producing enzyme, to obtain a treated food matrix; and
Processing the treated food matrix to produce an edible product having a meat-like flavor, said processing including an inactivation and/or a removal of the ROS producing enzyme.

2. Method according to claim 1, wherein said processing step comprises an inactivation heat treatment, a modification of pH and/or a modification of ionic strength of the treated food matrix.

3. Method according to claim 1, wherein the processing step comprises removal of the ROS producing enzyme.

4. Method according to claim 1, wherein the ROS producing enzyme is a synthetic ROS producing enzyme that produces at least one of the following reactive oxygen species: peroxides, superoxide, hydroxyl radicals, singlet oxygen, nitric oxide, peroxinitrite, free radicals, hypochlorite or hypochlorous acid, peroxyl radicals such as alkylperoxyl and hydroperoxyl, alkoxyl radicals, or combination thereof.

5. Method according to claim 1, wherein the step of treating the food matrix comprises adding the ROS producing enzyme at a concentration over 0.01 IU/g of the food matrix by wet weight.

6. Method according to claim 1, wherein the ROS producing enzyme is in contact with the food matrix during at most ten hours before being inactivated or removed during the processing step.

7. Method according to claim 1, wherein when treating the food matrix, said food matrix comprises at least 5% by weight of cultivated cells and/extracts thereof, from an organism of the Animalia kingdom excluding human, with respect to a wet weight of food matrix, said cultivated cells being selected from: stem cells, cells derived from differentiation of non-human embryonic stem cells; cells derived from differentiation of non-human induced pluripotent stem cells; cells derived from transdifferentiated non-human isolated cells; immortalized mature non-human cells; differentiated cells derived from differentiation of non-human progenitor cells, fibro-adipogenic progenitors, muscle cells, hepatocytes, fibroblasts, adipocytes, chondrocytes, keratinocytes, and combination thereof.

8. Method according to claim 1, wherein, prior to the step of treating, the food matrix is homogenized with fat, said fat comprising a non-human animal fat, a plant fat, a fermented fat or a mixture thereof.

9. Method according to claim 1, wherein when treating the food matrix, said food matrix comprises at least 5% of lipids by weight with respect to a wet weight of the food matrix.

10. Method according to claim 1, wherein when treating the food matrix, said food matrix comprises at least 1% by weight of unsaturated fatty acids, with respect to a wet weight of the food matrix.

11. Method according to claim 1, further comprising, after the step of treating the food matrix, a step of heat treatment until a core temperature of the food matrix of at least 50° C. is reached for at least 5 min.

12. An edible product having a meat-like flavor, produced from cultivated cells, said edible product comprising a treated food matrix, said treated food matrix having been treated with a reactive oxygen species (ROS) producing enzyme, said treated food matrix comprising cultivated cells and/or extracts thereof, said cultivated cells being cells from an organism of the Animalia kingdom excluding human, cells from an organism of the Bacteria kingdom and/or cells from an organism of the Fungi kingdom; and a reactive oxygen species producing enzyme.

13. An edible product according to claim 12, wherein the edible product comprises at least 1% by weight of the cultivated cells and/or extracts thereof, with respect to a total wet weight, and wherein the edible product further comprises the reactive oxygen species producing enzyme at a concentration of at least 0.0001 ng/g.

14. An edible product according to claim 12, wherein the edible product comprises the treated food matrix as an emulsion.

15. An edible product according to claim 12, wherein the edible product comprises at least 0.5% in weight of unsaturated fatty acids, with respect to a total wet weight of the edible product.

16. Method according to claim 5, said ROS producing enzyme being added at a concentration over 0.1 IU/g of the food matrix by wet weight.

17. An edible product according to claim 14, said emulsion being a microemulsion.

18. Method according to claim 1, wherein when treating the food matrix, said food matrix comprises at least 5% by weight of cultivated cells and extracts thereof, from an organism of the Animalia kingdom excluding human, with respect to a wet weight of said food matrix.

19. An edible product according to claim 12, wherein the edible product comprises at least 51% by weight of lipids with respect to a total wet weight of the edible product.

20. An edible product according to the claim 12, wherein the edible product comprises a food grade oil-in-water (O/W) or water-in-oil (W/O) emulsion or microemulsion.
